(19) United States
(12) Patent Application Publication
(10) Pub. No.: US 2026/0305954 A1
(43) Pub. Date: Oct. 8, 2026

(54) MULTI FUNCTIONAL HEALTH MONITORING METHOD BASED ON MILLIMETER WAVE RADAR

(71) Applicant: Tinglan Technology (Shenzhen) Co., Ltd., Shenzhen (CN)

(72) Inventors: Donghong Huang, Shenzhen (CN); Yuan Dong, Shenzhen (CN)

(73) Assignee: Tinglan Technology (Shenzhen) Co., Ltd., Shenzhen (CN)

(21) Appl. No.: 19/685,097

(22) Filed: May 22, 2026

Related U.S. Application Data
(63) Continuation of application No. PCT/CN2024/094482, filed on May 21, 2024.

(30) Foreign Application Priority Data
Nov. 23, 2023 (CN) 2023115805159

(51) Int. Cl. A61B 5/0205; A61B 5/00

(52) U.S. Cl. CPC A61B 5/0205; A61B 5/725; A61B 5/7257; A61B 5/7282; A61B 5/746; A61B 5/7465

(57) ABSTRACT

This application discloses a multifunctional health monitoring method, system, and computer-readable storage medium based on millimeter wave radar. The method comprises: receiving echo signals in space, and calculating the user's point cloud data and phase change data caused by the user's breathing and heartbeat based on the echo signals; Calculate user behavior based on the point cloud data provided; Calculate the user's vital signs based on the phase change data, including respiratory rate and heart rate; Determine that the user has triggered a set alarm event based on the behavior and/or vital signs, and execute the corresponding alarm operation based on the type of alarm event triggered.

### CROSS-REFERENCE TO RELATED APPLICATIONS

The application claims priority to Chinese patent application No. 2023115805159, filed o Nov. 23, 2023, the entire contents of which are incorporated herein by reference.

### TECHNICAL FIELD

This application relates to the field of radar monitoring technology, particularly to a multifunctional health monitoring method, system, and computer-readable storage medium based on millimeter wave radar.

### BACKGROUND

With the trend of aging population, the health and daily care of the elderly are becoming increasingly important. Health monitoring, accidental falls, and solitary outings require a large amount of manpower and material resources to support. Nowadays, there are many wearable and non wearable devices on the market to monitor the health, falls, and outings of the elderly.

Wearable devices are usually dominated by wristbands and bracelets. These wearable devices often have issues with uncomfortable wearing experience and inability to monitor around the clock.

Non wearable devices are usually dominated by cameras and radars. The problem with the camera solution is that it requires real-time recording of user activities during monitoring, which can easily lead to privacy violations. The problem with radar solutions is that they can usually only perform one of multiple monitoring functions such as fall detection and crowd detection, which leads to many limitations in their practical daily care.

### SUMMARY

The present embodiment provides a multifunctional health monitoring method based on millimeter wave radar, aiming to achieve all-weather, contactless, and privacy free multifunctional monitoring.

To achieve the above objectives, the embodiments of the present application provide a multifunctional health monitoring method based on millimeter wave radar, comprising:

Receive echo signals in space and calculate the user's point cloud data and phase change data caused by the user's breathing and heartbeat based on the echo signals;

Calculate user behavior based on the point cloud data provided;

Calculate the user's vital signs based on the phase change data, including respiratory rate and heart rate;

Determine that the user has triggered a set alarm event based on the behavior and/or vital signs, and execute the corresponding alarm operation based on the type of alarm event triggered.

It can be understood that the multifunctional health monitoring method based on millimeter wave radar in the technical solution of the present application can obtain point cloud data and phase change data of the monitored user through echo signals in space, and then calculate the behavior and vital signs of the monitored user based on the point cloud data and phase change data. In this way, multiple monitoring functions can be simultaneously implemented based on the behavior and vital signs, such as number detection, fall detection, heart rate detection, breathing detection, outdoor detection, etc., and these monitoring functions are all-weather and insensitive. Therefore, compared to existing monitoring schemes, this scheme has the following advantages:
1. Multi functional monitoring: The technical solution of this application is based on millimeter wave radar technology, which can simultaneously achieve multiple detection functions such as number detection, fall detection, heart rate detection, respiratory detection, and outdoor detection. Users can choose one or more of these functions according to their needs;
2. Low cost: The technical solution of this application only requires one radar to achieve multiple monitoring functions. Compared to a system that combines multiple non wearable devices to achieve multiple functional monitoring, the cost is lower and the cost-effectiveness is higher;
3. Non contact all-weather monitoring: The monitoring method of the technical solution in this application can achieve 24-hour all-weather monitoring without any contact, achieving true seamless protection;
4. Privacy protection: Unlike cameras, millimeter wave radar does not generate images during monitoring, but only captures simple trajectories and point cloud data. This means that users' privacy is effectively protected and will not touch upon personal privacy issues;
5. Two way communication: When the monitored user triggers an alarm event, the system can make a call to the bound terminal through the cloud server platform, so that the monitored user can contact the terminal user in the first time.

### BRIEF DESCRIPTION OF DRAWINGS

In order to provide a clearer explanation of the embodiments or technical solutions in the present application or existing technology, a brief introduction will be given to the accompanying drawings required for the description of the embodiments or existing technology. It is obvious that the accompanying drawings described below are only some embodiments of the present application. For those skilled in the art, other drawings can be obtained based on the structures shown in these drawings without creative labor.

FIG. 1 is a module structure diagram of an embodiment of a multifunctional health monitoring system based on millimeter wave radar in the present application;

FIG. 2 is a flowchart of an embodiment of a multifunctional health monitoring method based on millimeter wave radar in the present application.

The implementation, functional characteristics, and advantages of the purpose of this application will be further explained in conjunction with the embodiments and with reference to the accompanying drawings.

### DETAILED DESCRIPTION OF THE EMBODIMENTS

It should be understood that the specific embodiments described herein are only used to explain the present application and are not intended to limit the present application.

In order to better understand the above technical solution, the exemplary embodiments of the present disclosure will be described in more detail below with reference to the accompanying drawings. Although exemplary embodiments of the present disclosure are shown in the accompanying drawings, it should be understood that the present disclosure may be implemented in various forms and should not be limited by the embodiments described herein. On the contrary, these embodiments are provided to enable a more thorough understanding of this disclosure and to fully convey the scope of this disclosure to those skilled in the art.

It should be noted that any reference symbols located between parentheses should not be constructed as limitations on the claims. The use of “including” in the text does not exclude the existence of components or steps that are not listed in the claims. The quantity word ‘one’ or ‘one’ before the component does not exclude the existence of multiple such components. This application can be implemented by means of hardware comprising several different components and by means of appropriately programmed computers. In the unit claims listing several devices, several of these devices may be specifically embodied through the same hardware item. The use of words such as ‘first’, ‘second’, and ‘third’ does not indicate any order and can be interpreted as names.

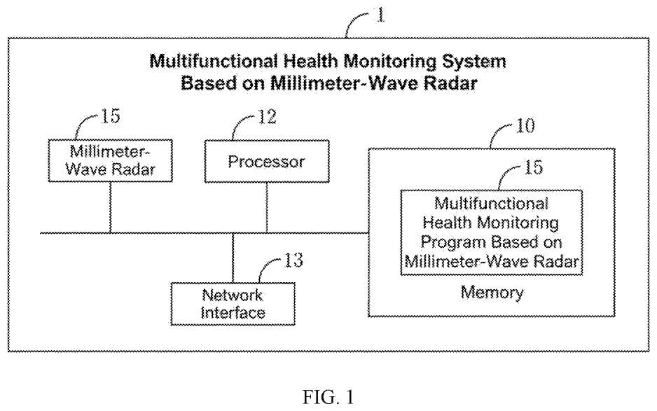

As shown in FIG. 1, FIG. 1 is a schematic diagram of the structure of a multifunctional health monitoring system based on millimeter wave radar according to an embodiment of the present application.

As shown in FIG. 1, the server 1 includes: millimeter wave radar 15, memory 11, processor 12, and network interface 13.

In this embodiment, the radar of the present application adopts TD-MIMO (Instant Division Multiple Access Multiple Input Multiple Output) signal transmission mode, transmitting a linear frequency modulated continuous wave signal (chirp signal). The radar signal is transmitted by the transmitting antenna, and the transmitted electromagnetic wave signal encounters obstacles and is reflected back. After time, the receiving antenna receives the echo signal, passes through a low-noise amplifier to filter out the noise influence, and then mixes with one of the transmitting signals. After passing through a low-pass filter, the intermediate frequency signal (IF signal) is obtained. The ADC signal can be obtained by digital sampling of the intermediate frequency signal. The frequency of the intermediate frequency signal is:, where k is the frequency modulation slope of the signal, is the target delay, and the delay is related to the target distance d as follows: For the target distance, c is the speed of light. Therefore, the distance between the target and the radar can be obtained from the frequency of the intermediate frequency signal. τf_{IF}f_{IF}=k*τ_{ττ} τ=2d/c f_{IF}=k*τ=k2d/c

Furthermore, the millimeter wave radar of this application adopts a design of 3 transmitting and 4 receiving antennas, with 3 transmitting antennas transmitting signals in a time-division multiplexing manner and 4 receiving antennas simultaneously receiving signals. Each transmitting antenna sends 32 chips in one frame, with 512 ADC sampling points per chip. I/Q complex sampling is used, and the amount of ADC data received in one frame is 12*32*512*2.

Optionally, there are two installation methods for the radar in this application, namely top mounted and side mounted. The top mounted radar is installed on the ceiling or suspended ceiling directly above the bed, while the side mounted radar is installed in the middle position above the bed head, within a range of 1.5 m to 1.8 m from the ground.

Furthermore, the memory 11 may also include internal storage units of the server 1 as well as external storage devices. Memory 11 can not only be used to store application software and various data installed on server 1, such as the code of the multifunctional health monitoring program 10 based on millimeter wave radar, but also temporarily store data that has been or will be output.

In some embodiments, processor 12 may be a Central Processing Unit (CPU), controller, microcontroller, microprocessor, or other data processing chip used to run program code stored in memory 11 or process data, such as executing a multifunctional health monitoring program 10 based on millimeter wave radar.

Network interface 13 can optionally include standard wired interfaces and wireless interfaces (such as WI-FI interfaces), typically used to establish communication connections between server 1 and other electronic devices.

The network can be Internet, cloud network, Wi Fi network, personal network (PAN), local area network (LAN) and/or metropolitan area network (MAN). Various devices in the network environment can be configured to connect to the communication network according to various wired and wireless communication protocols. Examples of such wired and wireless communication protocols may include, but are not limited to, at least one of the following: transmission control protocol and Internet protocol (TCP/IP), user datagram protocol (UDP), hypertext transmission protocol (HTTP), file transfer protocol (FTP), ZigBee, EDGE, IEEE 802.11, optical fidelity (Li Fi), 802.16, IEEE 802.11s, IEEE 802.11g, multi hop communication, wireless access point (AP), device to device communication, cellular communication protocol and/or Bluetooth communication protocol or a combination thereof.

Optionally, the server may also include a user interface, which may include a display, an input unit such as a keyboard, and optional user interfaces may also include standard wired and wireless interfaces. Optionally, in some embodiments, the display may be an LED display, a liquid crystal display, a touch sensitive liquid crystal display, and an OLED (Organic Light Emitting Diode) touch sensor, among others. Among them, the display can also be referred to as a screen or display unit, used to display the information processed in server 1 and to display the visual user interface.

FIG. 1 only shows server 1 with components 11-13 and a multifunctional health monitoring program 10 based on millimeter wave radar. Those skilled in the art can understand that the structure shown in FIG. 1 does not constitute a limitation on server 1, and may include fewer or more components than shown in the figure, or combine certain components, or arrange different components.

Based on the hardware architecture of the multifunctional health monitoring system based on millimeter wave radar mentioned above, this application proposes an implementation example of the multifunctional health monitoring method based on millimeter wave radar. The multifunctional health monitoring method based on millimeter wave radar in this application aims to achieve all-weather, contactless, and privacy free multifunctional monitoring.

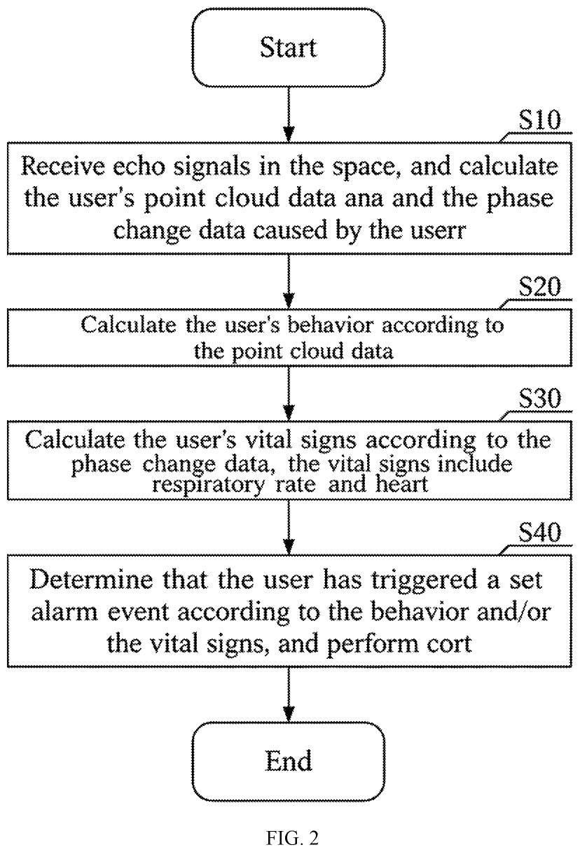

Referring to FIG. 2, FIG. 2 shows an embodiment of a multifunctional health monitoring method based on millimeter wave radar in the present application. The multifunctional health monitoring method based on millimeter wave radar includes the following steps:

S10. Receive echo signals in space and calculate the user's point cloud data and phase change data caused by the user's breathing and heartbeat based on the echo signals.

Among them, users refer to the entities that need to be monitored, followed, and protected. For example, the user can be one of the following: a patient, an elderly person, or a baby.

Point cloud data is a collection of three-dimensional coordinate points that describe the shape and position of an object or scene in three-dimensional space. Furthermore, due to the small movements of the chest and chest caused by human respiration and heartbeat, these movements can cause periodic phase changes in the echo signals. Based on this periodic phase change, the radar system can set certain filtering conditions to distinguish between users in space and stationary backgrounds, thereby accurately monitoring the phase change data caused by users' breathing and heartbeat.

Specifically, millimeter wave radar systems first send millimeter wave signals, which interact with objects in space and reflect back. The receiver receives these echo signals and uses their time delay and intensity information to calculate the user's point cloud data and phase change data.

S20. Calculate user behavior based on the point cloud data.

Specifically, behavior refers to the information of user behavior inferred by analyzing point cloud data obtained from millimeter wave radar. These data include detailed descriptions of users' activities and actions during the estimated time period. For example, behavior can include user posture data (such as standing, sitting, walking, running, stretching, etc.), motion trajectory data (direction, speed, and path of movement, etc.), posture stability data (such as monitoring whether the user is maintaining balance, shaking or swaying, etc.), etc.

Specifically, by analyzing the shape and position in the point cloud, the system can infer the user's current posture, such as standing, sitting, lying down, etc. In this way, the user's posture data can be obtained.

Meanwhile, by comparing point cloud data between consecutive frames, the system can analyze the user's movement trajectory, speed, and direction to obtain the user's trajectory data.

It is worth noting that point cloud data can also detect the number of people in the space to obtain the current number of people in the space.

S30. Calculate the user's vital signs based on the phase change data, including respiratory rate and heart rate.

Specifically, when the human body breathes or the heart beats, the movement of the chest and heart can cause changes in the phase of millimeter wave signals. This change is usually manifested as a phase shift or rate of change in the signal. The phase change caused by breathing usually has different frequencies and characteristics from the phase change caused by heartbeat. Breathing typically has a lower frequency (usually between 0.1 Hz and 0.8 Hz), while heartbeat typically has a higher frequency (usually between 0.8 Hz and 2.5 Hz or higher). Based on the above principles, non-contact vital sign monitoring can be achieved by monitoring the phase changes of echo signals caused by users' breathing and heartbeat, and calculating the respiratory frequency and/or heart rate of each user through methods such as fast Fourier transform.

It is worth noting that in some embodiments, the system can display the detected behaviors and vital signs in real-time on a terminal, which can be a mini program, mobile device app, webpage, etc.

S40. If it is determined based on the behavior and/or vital signs that the user has triggered a set alarm event, the corresponding alarm operation will be executed according to the type of alarm event triggered.

Specifically, the set alarm event refers to a series of specific situations or conditions predefined and configured in the radar system. When the user's behavior or vital signs meet these conditions, the system will trigger corresponding alarm notifications or emergency response measures. It is worth noting that these alarm events can be customized based on the user's health condition, medical standards, personal needs, or special circumstances.

For example, the set alarm events can include the following events: falls, prolonged immobility, abnormal breathing, abnormal heart rate, going out alone, etc.

Specifically, after obtaining behavior and vital signs, the radar system can monitor these point cloud data and vital signs in real time, and compare them with the set alarm threshold to monitor whether the user triggers the set alarm event.

In some embodiments, the system may monitor whether a user triggers an alarm event solely by comparing behavior with a set alarm threshold, or comparing vital signs with a set alarm threshold.

In other embodiments, the system can comprehensively consider behavior and vital signs, and by analyzing these data comprehensively, the system can more accurately monitor the user's health status. For example, if a user has been inactive for a long time (behavior) and has an abnormally low heart rate (vital signs), the system may monitor that the user may be in a dangerous state.

Furthermore, after determining that the user has triggered the set alarm event, the system can execute the corresponding alarm operation based on the type of alarm event triggered. Depending on the level of urgency and situation, the system may perform different actions to ensure user safety and provide appropriate assistance.

For example, based on the type of alarm event triggered by the user, the system can send different types of alarm information to pre bound terminals. For example, when the monitored user triggers a fall event, the system can send alarm information about the user's fall to the pre bound terminal through phone calls, text messages, application push notifications, emails, instant messages, etc., and remind the terminal user to take appropriate actions in a timely manner.

Among them, end users can be relatives, guardians, friends, etc. of the monitored user, as well as family doctors, medical institutions, rehabilitation institutions, etc. who have caregiving obligations with the monitored user.

In some embodiments, based on the alarm operation, the radar system of the technical solution of the present application can also preset a microphone and a speaker, thereby enabling the system to have bidirectional voice communication function. In this way, after the monitored user triggers an alarm event and the system performs an alarm operation, the system can actively initiate a communication request with the bound terminal, and after the communication is established, achieve two-way voice communication. In this way, users can receive timely communication and assistance, which helps to provide quick rescue or support in emergency situations.

It can be understood that the multifunctional health monitoring method based on millimeter wave radar in the technical solution of the present application can obtain point cloud data and phase change data of the monitored user through echo signals in space, and then calculate the behavior and vital signs of the monitored user based on the point cloud data and phase change data. In this way, multiple monitoring functions can be simultaneously implemented based on the behavior and vital signs, such as number detection, fall detection, heart rate detection, breathing detection, outdoor detection, etc., and these monitoring functions are all-weather and insensitive.

Therefore, compared to existing monitoring schemes, this scheme has the following advantages:
1. Multi functional monitoring: The technical solution of this application is based on millimeter wave radar technology, which can simultaneously achieve multiple detection functions such as number detection, fall detection, heart rate detection, respiratory detection, and outdoor detection. Users can choose one or more of these functions according to their needs;
2. Low cost: The technical solution of this application only requires one radar to achieve multiple monitoring functions. Compared to a system that combines multiple non wearable devices to achieve multiple functional monitoring, the cost is lower and the cost-effectiveness is higher;
3. Non contact all-weather monitoring: The monitoring method of the technical solution in this application can achieve 24-hour all-weather monitoring without any contact, achieving true seamless protection;
4. Privacy protection: Unlike cameras, millimeter wave radar does not generate images during monitoring, but only captures simple trajectories and point cloud data. This means that users' privacy is effectively protected and will not touch upon personal privacy issues;
5. Two way communication: When the monitored user triggers an alarm event, the system can make a call to the bound terminal through the cloud server platform, so that the monitored user can contact the terminal user in the first time.

In some embodiments, calculating the user's point cloud data based on the echo signal includes:

S11. Convert the echo signal into a digital signal.

Specifically, the echo signals returned by objects in space are analog signals, which need to be converted into digital signals through an analog-to-digital conversion module (ADC) before further processing.

S12. Perform Range FFT and Doppler FFT on the digital signal to obtain the Range Doppler map (RD map) of all target units in the space.

Among them, in radar signal processing, the distance dimension is used to measure the distance between the target object and the radar, and the Doppler dimension is used to measure the velocity of the target object.

Furthermore, Range FFT is a method of measuring target distance by utilizing the relationship between the frequency variation of the chirp signal and the target distance. Doppler FFT is a method of measuring target velocity by utilizing the relationship between the frequency change of the echo signal caused by target motion and the target velocity.

In this embodiment, the system first performs Range FFT on each antenna of the radar to obtain the distance information of each target unit in space. Perform a Doppler FFT on the results of the Range FFT to obtain Doppler dimensional data for each target unit in space. By combining the Range FFT results with the Doppler FFT results, the RD map can be obtained.

S13. Perform incoherent accumulation on the distance Doppler spectrum, and use preset threshold parameters to perform 2D Constant False Alarm Rate Detector (2D CFAR) on the accumulated results to extract the target units corresponding to the user on the distance Doppler spectrum.

Among them, after obtaining the RD map of each antenna, non coherent accumulation can be performed on the RD maps of all antennas to obtain the accumulated RD map. Non coherent accumulation is a method of adding the RD maps of different antennas or frames based on their amplitudes without considering phase differences. Non coherent accumulation can improve the signal-to-noise ratio of the target unit, making it easier to detect the target unit during the subsequent two-dimensional constant false alarm detection steps.

Furthermore, 2D CFAR detection is a method of constant false alarm detection in both distance and Doppler dimensions, which can determine the presence of a target unit based on the average value of each unit and surrounding units in the RD map.

Specifically, the preset threshold parameter is the false alarm rate threshold required for 2D CFAR detection. A two-dimensional threshold decision is made on the accumulated distance Doppler data, and targets corresponding to data points exceeding the threshold value are considered as existing targets. Therefore, after completing 2D CFAR detection, target units in space can be filtered out.

S14. Perform joint azimuth elevation angle measurement on the target unit corresponding to the user to obtain the azimuth and elevation angles of the target unit.

Among them, azimuth and elevation angles are parameters used to describe the position of the target unit in the radar detection space. They are angle measurements in polar coordinate systems used to determine the direction and elevation angle of the target object relative to the radar. The azimuth describes the horizontal direction of the target unit relative to the radar position. The pitch angle describes the vertical direction of the target unit relative to the radar position.

The azimuth elevation joint angle measurement algorithm is a method used to calculate the azimuth and elevation angles of a target object in the radar detection space. The core principle of the azimuth elevation joint angle measurement algorithm is to use multiple antennas or sensors to measure the signals of the target object, and combine the phase and amplitude information of these signals to calculate the azimuth and elevation angles.

Specifically, after selecting the target unit, all antenna data of the target unit can be extracted from the RD map. Then, the azimuth elevation joint angle measurement algorithm is used to calculate the azimuth and elevation angles of the target unit.

S15. Based on the azimuth and elevation angles, as well as the corresponding distance and Doppler units on the distance Doppler spectrum, obtain the point data of the target unit in space.

Specifically, after calculating the azimuth and elevation angles of the target unit, combined with the distance and Doppler units recorded in the RD map, the three-dimensional position of the target unit in the radar detection space can be determined, and then the point data of the target unit can be obtained.

S16. Generate point cloud data of the user in space based on all point data of the user.

Specifically, by analyzing the point data of all monitoring points of the user, the user's point cloud data can be obtained.

In this way, the point cloud data of the user can be extracted from the echo signal through steps S11 to S16 to achieve monitoring of the user's movement and position.

In some embodiments, calculating user behavior based on the point cloud data includes:

S21. Calculate the distribution status data of all monitoring points in the point cloud data.

Specifically, the system will analyze the user's point cloud data, including the location and density of monitoring points in space. These distribution status data can help the system understand which areas in space have monitoring points, as well as the distribution density of these monitoring points. By analyzing these features in point cloud data, the system can determine the user's current posture, such as standing or lying down.

S22. Cluster and fuse the point cloud data of the user to obtain the fitting center of the point cloud data.

Specifically, the user's point cloud data can be clustered and fused, and then the least squares method can be used for spherical fitting to obtain the centroid coordinates of the clustering results. These centroid coordinates can then be used as the user's fitting center in space.

Among them, spherical fitting is a mathematical method used to find the parameters of a sphere that are most suitable for a set of data points, including the center coordinates and radius of the sphere.

S23. Perform Kalman filtering tracking on the fitting center to obtain motion data of the fitting center, which includes motion trajectory data and motion velocity data.

Among them, Kalman filtering is an algorithm that uses linear system state equations to estimate the optimal state of a system through input-output observation data. By tracking the coordinates of the fitting center through Kalman filtering, the changes in the coordinates of the fitting center over time can be obtained.

Specifically, by tracking the coordinates of the fitting center through Kalman filtering, the coordinates of the fitting center at each sampling moment can be obtained, and the changes in the coordinates of the fitting center over time can be obtained, generating user movement trajectory data and velocity data.

S24. Generate the user's behavior based on the distribution state data and the motion data.

Specifically, in this step, the system will integrate distributed state data and motion data to generate user behavior. These behaviors can include the user's posture, movement trajectory, movement speed, dwell time, etc. By comprehensively analyzing these data, the system can identify user behavior patterns, such as walking, lying in bed, standing, sitting, etc.

It can be understood that using algorithms such as Kalman filtering to track the fitting center results in motion data with high accuracy. Meanwhile, point cloud data typically contains a certain degree of noise, and through clustering and fitting center calculations, the impact of noise on motion data calculations can be reduced. In addition, by combining the distribution status of cloud data with the motion data of the fitting center, user behavior is generated simultaneously, increasing the data dimension and further improving the accuracy of user behavior judgment.

In some embodiments, the set alarm events include fall events;

Monitor whether the user has triggered the set alarm event based on the behavior, including:

S411. Calculate the ratio of monitoring points in the point cloud data that are lower than or equal to the set height to all monitoring points based on the distribution state data.

Specifically, based on the user's point cloud data, the system will analyze the number of monitoring points in the point cloud data that are lower than or equal to the set height, and calculate the ratio of these monitoring points to all monitoring points. This ratio reflects the degree of contact between the user's body and these beds, floors, and other furniture.

For example, if the set height is lower than the height of the bed, the higher the proportion of monitoring points in the point cloud data that are lower than or equal to the set height, the closer the user is to the ground.

S412. Calculate the user's speed change value based on the motion speed data.

Specifically, based on the motion data, the system can calculate the speed change value of the user between adjacent frames, which represents the degree of change in the user's motion speed within a set time. This speed change value can be used to monitor the user's movement status, such as whether there are sudden rapid movements or stops.

S413. According to the motion trajectory data, it is confirmed that the user's action state is bedridden, and the speed change value is greater than or equal to the set value. When the ratio is greater than or equal to the set ratio, it is determined that the user has triggered a falling bed event.

Specifically, the system can confirm the user's action status based on their motion trajectory data, For example, when the user's movement trajectory remains within the range of the bed, it can be determined that the user is in a bedridden state.

Specifically, if the system detects that the user is in a bedridden state with a speed change value greater than or equal to the set threshold, and the proportion of monitoring points below or equal to the set height is greater than or equal to the set ratio, the system will determine that the user has triggered a bed fall event.

In some embodiments, the set alarm event includes a falling bed event.

Monitor whether the user has triggered the set alarm event based on the behavior, including:

S421. Calculate the ratio of monitoring points in the point cloud data that are lower than or equal to the set height to all monitoring points based on the distribution state data.

Specifically, based on the user's point cloud data, the system will analyze the number of monitoring points in the point cloud data that are lower than or equal to the set height, and calculate the ratio of these monitoring points to all monitoring points. This ratio reflects the degree of contact between the user's body and these beds, floors, and other furniture.

For example, if the set height is lower than the height of the bed, the higher the proportion of monitoring points in the point cloud data that are lower than or equal to the set height, the closer the user is to the ground.

S422. Calculate the user's speed change value based on the motion speed data.

Specifically, based on the motion data, the system can calculate the speed change value of the user between adjacent frames, which represents the degree of change in the user's motion speed within a set time. This speed change value can be used to monitor the user's movement status, such as whether there are sudden rapid movements or stops.

S423. According to the motion trajectory data, it is confirmed that the user's action state is bedridden, and the speed change value is greater than or equal to the set value. When the ratio is greater than or equal to the set ratio, it is determined that the user has triggered a falling bed event.

Specifically, the system can confirm the user's action status based on their motion trajectory data. For example, when the user's movement trajectory remains outside the range of the bed, it can be determined that the user is in an off bed state.

Specifically, if the system detects that the user is in an off bed state with a speed change value greater than or equal to the set threshold, and the proportion of monitoring points below or equal to the set height is greater than or equal to the set ratio, the system will determine that the user has triggered a fall event.

Through the above steps, multi-dimensional data such as height information, motion speed, and motion trajectory in point cloud data can be combined to accurately monitor whether users have triggered falling events or bed falling events, providing reliable basis for emergency detection and alarm.

In some embodiments, the set alarm events include solo outings.

Specifically, solitary outings refer to the behavior of monitored users leaving their homes, medical institutions, or other safe spaces alone without anyone else accompanying them. This type of event may pose risks or safety issues in specific contexts, especially for populations that require special attention or monitoring, such as the elderly, children, individuals with cognitive impairments or other illnesses (such as autism, epilepsy, etc.).

In some embodiments, monitoring whether the user has triggered a set alarm event based on the behavior includes:

When it is detected based on the motion trajectory data that the user has left the space and has not returned to the space after the first set time interval, it is determined that the user has triggered a solo outing event.

Specifically, when facing the need for independent outdoor monitoring, the boundary of the monitoring space can be set in the system. In this way, when the user's movement trajectory detects that they have crossed these boundaries, the system can determine that the user has left the space.

Furthermore, when the user leaves the room, the system will record the time when the user left the space and monitor whether the user returns to the space within the set time. If the user who left the space does not return within the set time, the system will determine that the user has triggered a solo outing event.

Once the system determines that the user has triggered a solo outing event, it will immediately trigger the corresponding alarm operation. This can include sending alert notifications to guardians, family members, or relevant agencies so that they can take appropriate action, such as contacting the user, finding the user's location, or notifying the police.

Through the above steps, the system can promptly detect and alert users when they go out alone, ensuring the safety of the monitored users.

In some embodiments, the set alarm events also include unmanned activity events.

Specifically, unmanned activity incidents refer to the behavior of monitored users who stay alone in their homes, medical institutions, or other safe spaces for a duration exceeding the set time without the accompaniment of others. This type of event may pose risks or safety issues in specific contexts, especially for populations that require special attention or monitoring, such as the elderly, children, individuals with cognitive impairments or other illnesses (such as autism, epilepsy, etc.).

In some embodiments, monitoring whether the user has triggered a set alarm event based on the behavior further includes:

When the duration of a user's single stay in space exceeds the second set duration detected based on the motion trajectory data, it is determined that the user has triggered an unmanned activity event.

Specifically, when faced with the need for long-term monitoring, the boundaries of the monitoring space can be set in the system. In this way, when the monitored user's movement trajectory detects that the user has not crossed these boundaries for a long time (referring to a duration greater than the second set time, such as more than 24 hours, 36 hours, etc.), the system can determine that the user has stayed in space for too long. Furthermore, the system will determine that the monitored user has triggered an unmanned activity event.

Once the system determines that the monitored user has triggered an unmanned activity event, it will immediately trigger the corresponding alarm operation. This can include sending alert notifications to guardians, family members, or relevant agencies so that they can take appropriate action, such as contacting the user, finding the user's location, or notifying the police.

Through the above steps, the system can promptly detect and alert the monitored user when they stay in the space for a long time, ensuring the safety of the monitored user.

In some embodiments, calculating the phase change data caused by the user's breathing and heartbeat based on the echo signal includes:

S110. After obtaining the distance Doppler spectra of all target units in space, extract phase data with Doppler 0 from the distance Doppler spectra of any antenna.

Specifically, the distance Doppler spectra of all target units in this space can be obtained in the step of acquiring the user's point cloud data. This approach can reduce the system's computational flow and save computing power and energy.

Furthermore, in a multi-channel millimeter wave radar system, the range Doppler spectrum of any antenna can be selected for data processing. In the selected distance Doppler map, the point with a Doppler frequency of 0 corresponds to a static or slowly moving target object (person or object), and the phase data of the target object can change with the small motion of the target object.

Therefore, by extracting phase data with Doppler frequency of 0, it can be used to screen out static (usually sleep state) users in space.

S120. Accumulate multiple frames of phase data to obtain slow Doppler phase data with Doppler 0.

Among them, slow Doppler refers to the frequency variation of the echo signal caused by the small motion of the target.

Specifically, the system will process phase data from multiple frames (such as 10 consecutive seconds) and calculate the differences between the multiple frames to obtain slow Doppler phase data for the target object with Doppler 0. In this way, the temporal changes of these data can be obtained.

It can be understood that by selecting data with Doppler 0 and accumulating multiple frames of data on them. By overlaying multiple frames of data, the observation time of the target object can be increased and the reliability of detection can be improved.

S130. Perform static filtering on the slow Doppler phase data to select slow Doppler phase data of users who are in a static state in space.

Specifically, by performing static filtering on slow Doppler dimensional data (subtracting the average of multiple frames from each frame), the stability of the background signal can be utilized to remove completely stationary backgrounds, thereby retaining only signals with changes. The objects corresponding to these changing signals can be regarded as static users in space. Correspondingly, these changing signals are the slow Doppler phase data of static users required for subsequent step calculations.

S140. Filter the peak values from the static filtered slow Doppler phase data based on a preset peak threshold, and determine the target distance unit of the static user based on the peak values.

Specifically, after performing static filtering, the system will filter out the peaks in the slow Doppler phase data based on the preset peak threshold. These peaks represent the user's breathing and heartbeat signals. By identifying these peaks, the system can determine the target distance unit where the static user is located.

S150. Extract phase from the target distance unit of the slow Doppler phase data to obtain the phase change data.

Finally, by extracting phase data from the determined target distance unit, the phase change data caused by the user's breathing and heartbeat can be obtained.

In some embodiments, calculating the user's vital signs based on the phase change data includes:

S311. Using a 6th order Butterworth filter designed based on the frequency range of respiratory rate, the phase change data is filtered to extract respiratory waveform data

Among them, the Butterworth filter is a digital filter commonly used in signal processing, which enhances the signal within a specific frequency range while suppressing unwanted frequency components. The 6th order Butterworth filter designed through the frequency range of respiratory frequency can emphasize respiratory waveform signals and reduce noise.

Specifically, after filtering the phase change data, the obtained signal can be considered as respiratory waveform data, which reflects the periodic respiratory changes of the target object.

S312. Perform fast Fourier transform on the respiratory waveform data to extract the respiratory cycle and calculate the user's respiratory frequency.

Specifically, by performing Fast Fourier Transform (FFT) on the extracted respiratory waveform data, the frequency components of the target object's respiratory cycle can be extracted from the waveform. Based on the periodic changes in the frequency components of the respiratory cycle, the respiratory frequency of the target object can be calculated.

In addition, by analyzing the results of FFT, the amplitude and phase information corresponding to the respiratory frequency in the spectrum can also be extracted. Among them, the amplitude information of respiratory rate can be used to represent the intensity of breathing.

In some embodiments, calculating the user's vital signs based on the phase change data includes:

S321. Using a 6th order Butterworth filter designed based on the frequency range of heart rate to filter the phase change data and extract heart rate waveform data.

Among them, the Butterworth filter is a digital filter commonly used in signal processing, which enhances the signal within a specific frequency range while suppressing unwanted frequency components. The 6th order Butterworth filter designed through the frequency range of heart rate can emphasize the heartbeat waveform signal and reduce noise.

Specifically, after filtering the phase change data, the obtained signal can be considered as heart rate waveform data, which reflects the periodic changes in heart rate of the target object.

S322. Perform wavelet transform on the heart rate waveform data and then perform fast Fourier transform to extract the heart rate cycle, and calculate the user's heart rate.

Among them, wavelet transform is a transformation that can decompose signals into basis functions of different scales and frequencies, used to extract detailed features of signals or remove noise.

Specifically, by performing wavelet transform on the extracted respiratory waveform data and performing fast Fourier transform (FFT), the frequency components of the target object's heart rate cycle can be extracted from the waveform. Based on the periodic changes in the frequency components of the heart rate cycle, the heart rate of the target object can be calculated.

In addition, by analyzing the results of FFT, amplitude and phase information corresponding to the spectral center rate can also be extracted. Among them, the amplitude information of heart rate can be used to represent the intensity of heartbeat.

In some embodiments, the set alarm events also include respiratory abnormality events and heart rate abnormality events.

Specifically, in addition to falling incidents, falling from bed incidents, and going out alone incidents, the system can also set respiratory and heart rate abnormal events as alarm events. Among them, respiratory abnormalities can include abnormal respiratory rate, respiratory pauses, etc; Abnormal heart rate events can include abnormal heart rate frequency, arrhythmia, or arrhythmia.

In some embodiments, monitoring whether the user has triggered a set alarm event based on the vital signs further includes:

S210. If the user's heart rate is detected to exceed the set heart rate range, it is determined that the user has triggered an abnormal heart rate event;

S220. If the user's respiratory rate is detected to exceed the set respiratory range, it is determined that the user has triggered a respiratory abnormality event.

Specifically, the system will monitor the user's vital signs (heart rate and respiratory rate) in real-time and set specific heart rate and respiratory rate ranges. If the system detects that the user's heart rate exceeds the set heart rate range, it determines that the user has triggered an abnormal heart rate event. Similarly, if the user's respiratory rate is detected to exceed the set respiratory range, the system determines that the user has triggered a respiratory abnormality event.

For example, if the user's heart rate exceeds the set upper limit, the system will determine it as an abnormal heart rate event. Excessive heart rate may indicate that the user is in a state of cardiovascular stress and needs to be vigilant.

For example, if the user's heart rate falls below the set lower limit, it is also considered an abnormal heart rate event. A slow heart rate may indicate that the user may have arrhythmia or other heart problems.

For example, if the user's respiratory rate exceeds the set upper limit, the system will determine it as a respiratory abnormality event. Excessive breathing may indicate that the user may be in a state of anxiety, respiratory illness, or other emergency.

For example, if the user's respiratory rate falls below the set lower limit, it is also considered a respiratory abnormality event. Slow breathing may indicate that the user may have respiratory problems or other potential health risks.

It is worth noting that when the system detects that the user's heart rate or respiratory rate exceeds the set range, it will immediately trigger corresponding alarm operations to take timely medical measures or notify medical personnel, guardians, etc., ensuring that the user can receive timely help and treatment when facing health risks.

In addition, the present embodiment also proposes a computer-readable storage medium, which can be a hard disk, multimedia card, SD card, flash memory card SMC. Any one or any combination of read-only memory (ROM), erasable programmable read-only memory (EPROM), portable compact disc read-only memory (CD-ROM), USB memory, etc. The computer-readable storage medium includes a multifunctional health monitoring program 10 based on millimeter wave radar. The specific implementation of the computer-readable storage medium in this application is roughly the same as the above-mentioned multifunctional health monitoring method based on millimeter wave radar and the specific implementation of server 1, and will not be repeated here.

Technicians in this field should understand that the embodiments of the present application can be provided as methods, systems, or computer program products. Therefore, the present application may take the form of a fully hardware implementation, a fully software implementation, or a combination of software and hardware implementation. Moreover, the present application may take the form of a computer program product implemented on one or more computer usable storage media (including but not limited to disk storage, CD-ROM, optical storage, etc.) containing computer usable program code.

This application is described with reference to the flowchart and/or block diagram of the method, device (system), and computer program product according to the embodiments of this application. It should be understood that each process and/or block in the flowchart and/or block diagram, as well as the combination of processes and/or blocks in the flowchart and/or block diagram, can be implemented by computer program instructions. These computer program instructions can be provided to a processor of a general-purpose computer, specialized computer, embedded processor, or other programmable data processing device to generate a machine, such that the instructions executed by the processor of the computer or other programmable data processing device generate a device for implementing the functions specified in one or more processes in the flowchart and/or one or more boxes in the block diagram.

These computer program instructions can also be stored in computer-readable memory that can guide a computer or other programmable data processing device to operate in a specific manner, causing the instructions stored in the computer-readable memory to produce a manufactured product including instruction devices that implement the functions specified in a flowchart or multiple flowcharts and/or a block diagram or multiple boxes.

These computer program instructions can also be loaded onto a computer or other programmable data processing device, enabling a series of operational steps to be executed on the computer or other programmable device to generate computer implemented processing. The instructions executed on the computer or other programmable device provide steps for implementing the functions specified in one or more processes in the flowchart and/or one or more boxes in the block diagram.

Although the preferred embodiments of the present application have been described, those skilled in the art may make additional changes and modifications to these embodiments once they have knowledge of the basic inventive concept. Therefore, the attached claims are intended to be interpreted as including preferred embodiments and all changes and modifications falling within the scope of the present application.

Obviously, technicians in this field can make various modifications and variations to this application without departing from its spirit and scope. In this way, if these modifications and variations of the present application fall within the scope of the claims and equivalent technologies of the present application, then the present application also intends to include these modifications and variations.

What is claimed is:

1. A multifunctional health monitoring method based on millimeter wave radar, comprising:
Receive echo signals in space and calculate the user's point cloud data and phase change data caused by the user's breathing and heartbeat based on the echo signals;
Calculate user behavior based on the point cloud data provided;
Calculate the user's vital signs based on the phase change data, including respiratory rate and heart rate;
Determine that the user has triggered a set alarm event based on the behavior and/or vital signs, and execute the corresponding alarm operation based on the type of alarm event triggered.

2. The multifunctional health monitoring method based on millimeter wave radar as claimed in claim 1, wherein calculating the user's point cloud data based on the echo signal comprises:
Convert the echo signal into a digital signal;
Perform distance dimension fast Fourier transform and Doppler dimension Fourier transform on the digital signal to obtain the distance Doppler spectra of all target units in the space;
Perform incoherent accumulation on the distance Doppler spectrum, and use preset threshold parameters to perform two-dimensional constant false alarm detection on the accumulated results, extracting the target unit corresponding to the user on the distance Doppler spectrum;
Perform joint azimuth elevation angle measurement on the target unit corresponding to the user to obtain the azimuth and elevation angles of the target unit;
Obtain point data corresponding to the target unit in space based on the azimuth and elevation angles, as well as the corresponding distance units on the range Doppler spectrum; and
Generate point cloud data for the user based on all their point data.

3. The multifunctional health monitoring method based on millimeter wave radar as claimed in claim 2, wherein calculating user behavior based on the point cloud data comprises:
Calculate the distribution status data of all monitoring points in the point cloud data;
Cluster and fuse the point cloud data of the user to obtain the fitting center of the point cloud data;
Perform Kalman filtering tracking on the fitting center to obtain motion data of the fitting center, which includes motion trajectory data and motion velocity data; and
Generate the user's behavior based on the distribution state data and the motion data.

4. The multifunctional health monitoring method based on millimeter wave radar as claimed in claim 3, wherein clustering and fusion are performed on the point cloud data of the user to obtain the fitting center of the point cloud data, comprising:
After clustering and fusing the user's point cloud data, the least squares method is used for spherical fitting to obtain the centroid coordinates of the clustering results, which are used as the fitting center of the point cloud data.

5. The multifunctional health monitoring method based on millimeter wave radar as claimed in claim 3, wherein the set alarm events include fall events;
Determine based on the behavior that the user has triggered the set alarm event, including:
Calculate the ratio of monitoring points in the point cloud data that are lower than or equal to the set height to all monitoring points based on the distribution state data;
Calculate the user's speed change value based on the motion speed data;
According to the motion trajectory data, if the user's action state is confirmed to be bedridden, and the speed change value is greater than or equal to the set value, and the ratio is greater than or equal to the set ratio, it is determined that the user has triggered a falling bed event.

6. The multifunctional health monitoring method based on millimeter wave radar as claimed in claim 3, wherein the set alarm event includes a falling bed event;
Determine based on the behavior that the user has triggered the set alarm event, including:
Calculate the ratio of monitoring points in the point cloud data that are lower than or equal to the set height to all monitoring points based on the distribution state data;
Calculate the user's speed change value based on the motion speed data;
According to the motion trajectory data, if the user's action state is confirmed to be off bed, and the speed change value is greater than or equal to the set value, and the ratio is greater than or equal to the set ratio, it is determined that the user has triggered a fall event.

7. The multifunctional health monitoring method based on millimeter wave radar as claimed in claim 3, wherein the set alarm events include solo outings;
Determine based on the behavior that the user has triggered the set alarm event, including:
According to the motion trajectory data, if the user leaves the space and does not return to the space after the first set time interval, it is determined that the user has triggered a solo outing event.

8. The multifunctional health monitoring method based on millimeter wave radar as claimed in claim 7, wherein detecting the user leaving the space based on the motion trajectory data comprises:
Set the boundary of the spatial area;
If it is detected through the motion trajectory data that the user has crossed the boundary of the area, it is determined that the user has left the monitoring space.

9. The multifunctional health monitoring method based on millimeter wave radar as claimed in claim 3, wherein the set alarm events include unmanned activity events;
Based on the behavior, it is determined that the user has triggered a set alarm event, which includes: if the user's single stay in the space exceeds the second set duration based on the motion trajectory data, it is determined that the user has triggered an unmanned activity event.

10. The multifunctional health monitoring method based on millimeter wave radar as claimed in claim 9, wherein the monitoring of the user's single stay in space exceeding a second set duration based on the motion trajectory data comprises:
Set the boundary of the spatial area;
If it is detected through the motion trajectory data that the user has not crossed the boundary of the area for a period of time exceeding the second set duration, it is determined that the user has stayed in the space for a single time exceeding the second set duration.

11. The multifunctional health monitoring method based on millimeter wave radar as claimed in claim 2, wherein the millimeter wave radar has multiple antennas;
Calculate the phase change data caused by the user's breathing and heartbeat based on the echo signal, including:
After obtaining the distance Doppler spectra of all target units in space, extract phase data with Doppler 0 from the distance Doppler spectra of any antenna;
Accumulate multiple frames of phase data to obtain slow Doppler phase data with Doppler 0;
Perform static filtering on the slow Doppler phase data to select slow Doppler phase data of users who are in a static state in space;
Select peak values from the static filtered slow Doppler phase data based on a preset peak threshold, and determine the target distance unit where the static user is located based on the peak values; and
Extract phase from the target distance unit of the slow Doppler phase data to obtain the phase change data.

12. The multifunctional health monitoring method based on millimeter wave radar as claimed in claim 1, wherein calculating the user's vital signs based on the phase change data comprises:
Using a 6th order Butterworth filter designed based on the frequency range of respiratory rate to filter the phase change data and extract respiratory waveform data;
Perform fast Fourier transform on the respiratory waveform data to extract the respiratory cycle and calculate the user's respiratory frequency.

13. The multifunctional health monitoring method based on millimeter wave radar as claimed in claim 1, wherein calculating the user's vital signs based on the phase change data comprises:
Using a 6th order Butterworth filter designed based on the frequency range of heart rate to filter the phase change data and extract heart rate waveform data;
Perform wavelet transform on the heart rate waveform data and then perform fast Fourier transform to extract the heart rate cycle, and calculate the user's heart rate.

14. The multifunctional health monitoring method based on millimeter wave radar as claimed in claim 1, wherein the set alarm events include abnormal heart rate events;
Determine that the user has triggered a set alarm event based on the vital signs, including:
Detected that the user's heart rate exceeded the set heart rate range, determining that the user triggered an abnormal heart rate event.

15. The multifunctional health monitoring method based on millimeter wave radar as claimed in claim 1, wherein the set alarm events include respiratory abnormality events;
Determine that the user has triggered a set alarm event based on the vital signs, including:
Detected that the user's breathing rate exceeded the set breathing range, determining that the user triggered a breathing abnormality event.

16. The multifunctional health monitoring method based on millimeter wave radar as claimed in claim 1, wherein corresponding alarm operations are executed according to the triggered alarm event type, including:
Detecting a user triggering a fall event, send an alarm message for the user's fall to the pre bound terminal through at least one of the following methods:
Phone, SMS, app push, email, instant messaging.

17. The multifunctional health monitoring method based on millimeter wave radar as claimed in claim 1, wherein the method further comprises:
After the monitored user triggers an alarm event and performs an alarm operation, a communication request is initiated with the bound terminal, and a two-way voice call is achieved after the communication is established.

18. A multifunctional health monitoring system based on millimeter wave radar, comprising a millimeter wave radar, a memory, a processor, and a millimeter wave radar based multifunctional health monitoring program stored on the memory and executable on the processor; When the processor executes the millimeter wave radar based multifunctional health monitoring program, it implements the millimeter wave radar based multifunctional health monitoring method according to claim 1.

19. A computer-readable storage medium, wherein a multifunctional health monitoring program based on millimeter wave radar is stored on the computer-readable storage medium, and when the multifunctional health monitoring program based on millimeter wave radar is executed by a processor, the multifunctional health monitoring method based on millimeter wave radar as claimed in claim 1 is implemented.
